(19) United States
(12) Patent Application Publication
(10) Pub. No.: US 2017/0036387 A1
(43) Pub. Date: Feb. 9, 2017

(54) FACILITY FOR PRODUCING CONTAINERS, COMPRISING A DEVICE FOR DISINFECTING A TRANSFER WHEEL

(71) Applicant: SIDEL PARTICIPATIONS, Octeville-sur-Mer (FR)

(72) Inventors: Christophe MONDOLO, Octeville-sur-Mer (FR); Jerome DEMARE, Octeville-sur-Mer (FR); Sandy LETELLIER, Octeville-sur-Mer (FR)

(21) Appl. No.: 15/304,651

(22) PCT Filed: Apr. 2, 2015
(86) PCT No.: PCT/EP2015/057410

§ 371 Date: Oct. 17, 2016

(30) Foreign Application Priority Data
Apr. 17, 2014 (FR) 1453491

(51) Int. Cl. B29C 49/46; B65B 7/16; A61L 2/22; B65B 43/50; B65B 65/02; A61L 2/24; B29C 49/42; B65B 3/02

(52) U.S. Cl. CPC B29C 49/46; B29C 49/421; B29C 49/4236; B65B 7/16; B65B 3/022; B65B 43/50; B65B 65/02; A61L 2/24; A61L 2/22; B29C 2049/4697; B29L 2031/7158

(57) ABSTRACT

A facility (10) for producing containers (12), especially bottles, includes a device (100) for disinfecting a transfer wheel (30), which is controlled from outside the enclosures (14, 15) of the facility (10), for the disinfection of at least the upper container (12)—transfer part (30A) of the wheel (30), which occupies a retracted position, by spraying at least one disinfecting agent.

This invention relates to a facility for producing containers, comprising a device for disinfecting a transfer wheel.

This invention relates more particularly to a facility for producing containers, in particular bottles, comprising at least:
A first protective enclosure delimiting a first zone inside of which is arranged at least one unit for blow molding containers,
A second confinement enclosure that, at least in the part attached to the first adjacent enclosure by a common part, delimits a second sterile zone inside of which is arranged at least one unit for filling manufactured containers,
At least one opening that, made in said common part of said first and second enclosures, is designed to make possible the transfer of the containers from the blow-molding unit to the filling unit,
Means for sealing said opening that are able to be moved selectively between an open position in which the sealing means allow said transfer of the containers through the opening and a closed position in which the sealing means prevent any transfer by insulating the second enclosure to carry out a decontamination of the filling unit,
A transfer device that comprises at least one transfer wheel that is adjacent to said opening for transferring the containers between the blow-molding unit and the filling unit, with said transfer wheel comprising at least one upper part for transfer of the containers that is mounted to move between at least a first transfer position that is occupied when the facility is in a production mode and a second retracted position that is occupied when the facility is in a decontamination mode, with the movement of said upper part of the wheel mounted to move between said transfer and retracted positions being controlled from outside the facility, and
A drive system of the transfer wheel for driving in rotation at least the upper part for transfer of the containers from the wheel when, in the production mode, said upper part occupies said transfer position.

The document WO-2010/081759 describes and shows an example of such a facility for producing containers, in particular bottles.

The production facility is used for the manufacturing of containers made of plastic material, in particular polyethylene terephthalate (PET).

The containers are generally manufactured by successively initiating the thermal packaging of preforms made of plastic material in a furnace, then in the transformation of the preforms into bottles in a blow-molding unit, with the shaping being done by, for example, blow molding or by stretch blow molding in a mold, and finally in the transfer of the containers that are obtained toward a unit for filling and capping.

The facility thus comprises, upstream from the filling unit, a manufacturing unit that preferably comprises a thermal conditioning furnace and a blow-molding unit.

Generally, the filling unit and the manufacturing unit, at the very least the blow-molding unit, are juxtaposed to obtain a production facility that is compact and integrally carries out the process for producing the containers until the finished products are obtained.

As explained in the document WO-2010/081759, it is sought in such a production facility to reduce by any means the risks of contamination of the containers, which containers are, moreover, able to be filled with products that are more or less sensitive to such risks.

Consequently, it is known to implement different actions there for the sole purposes of monitoring and controlling the microbiological quality of the production environment, in particular the elimination of pathogenic agents, such as germs, spores, bacteria, etc., which may affect the product that is contained in the containers by making it in particular unsuitable for consumption.

To do this, the actions aim at not only the decontamination of containers but also those of the preforms from which they are manufactured as well as that of the facility itself in general.

The documents according to the state of the art: WO-2006/136498; WO-2008/049876; FR-2,915,127 constitute nonlimiting examples that illustrate such actions, and reference will advantageously be made for more ample details in each of these documents, cited, moreover, in the preamble of the document WO-2010/081759.

Of course, these different examples of actions may be simultaneously implemented in the same facility for drastically reducing the risks of contamination.

In the production process, the operation for filling the container is usually recognized as being the most sensitive relative to the risks of contamination.

The containers introduced into the filling unit, however, are only one of the primary carriers of contamination.

Actually, pathogenic agents, from the moment that they are present in the direct environment of the containers, from the air to the elements of the units of the facility, in particular may contaminate the inside volume of the container.

This is the reason for which, in addition to the sterilization or disinfection treatments whose object is directly the product intended to be introduced into the container and the container itself, decontamination of the filling unit is also initiated by chemical means, in particular by spraying sterilizing solutions such as sodium hydroxide (NaOH) or hydrogen peroxide (H₂O₂).

The decontamination of the filling unit corresponds to an operating mode, a so-called decontamination mode, of the facility that precedes and/or succeeds the implementation of the operating mode, the so-called production mode, of the facility in which the containers are manufactured.

To be able to initiate the implementation of the mode for decontamination of the filling unit, it is necessary to insulate the filling unit from the rest of the facility and most particularly from the adjacent blow-molding unit.

Actually, although the elements of the filling unit are made in appropriate materials, such as 316L stainless steel, to withstand the chemical attacks from the above-mentioned sterilizing solutions that are used for decontamination, this is not, however, the case of the blow-molding unit, nor generally of the adjacent transfer device inserted between these units.

Thus, the sterilizing solutions that are used for the decontamination may bring about undesirable chemical attacks, in particular the corrosion of elements of the blow-molding unit, such as the molds, as well as that of elements of the transfer device.

The document WO-2010/081759 describes the implementation of the decontamination of the filling unit and describes most particularly the risks of contamination linked to the intervention by an operator entering the first enclosure to carry out operations there involving the disassembly and/or remounting of at least one part of the transfer wheel.

Such disassembly and/or remounting operations of at least one part of the transfer wheel of the containers are required for making possible the closing by sealing means of the communication opening through which said part extends from the wheel for ensuring the transfer of the containers into the production mode.

In the facility according to the document WO-2010/081759, said communication opening is made in a common part between the first enclosure comprising the blow-molding unit and the second enclosure comprising the filling unit, with said sealing means making it possible to selectively insulate the filling unit during decontamination.

As explained in this document, the operator constitutes a significant potential carrier for introduction of pathogenic agents inside the facility creating risks of contamination of different orders and multiples.

To remedy this, the document WO-2010/081759 proposes a new design of the facility and of the transfer wheel according to which the retraction of the upper part for transfer of the containers from the wheel, which extends through the opening in the production mode, may be carried out by an operator from outside the facility.

Owing to this new design, the operator remains outside the facility, without ever physically entering into the first enclosure for carrying out, manually or automatically, the movement of at least the upper part for transfer of the containers from the wheel between a transfer position and a retracted position.

Advantageously, the operator that acts remotely cannot at any time form a carrier for introducing pathogenic agents inside the volume of the facility delimited by the enclosures, in such a way that the above-mentioned risks of contamination are eliminated.

In addition, such a design also makes it possible to replace the operations of disassembly and remounting that were previously carried out by simpler and faster operations that make it possible to achieve the retraction of at least the upper part for transfer of the containers from the wheel and the closing of the opening by sealing means so as to decontaminate the filling unit.

The implementation of the decontamination by chemical means of the filling unit with the new design of the transfer wheel described in the document WO-2010/081759 is an example of action that further enhances food safety and thus meets the demand for ever tighter monitoring and control of the microbiological quality of the container production environment, in particular the elimination of pathogenic agents, such as germs, spores, bacteria, etc.

According to the above-mentioned document FR-2,915,127, it is known to equip the facility with a system for blowing in filtered air into the enclosures for establishing there an overpressure that is suitable for limiting the risks of contamination of the preforms in the exit zone of the furnace, such as manufactured containers.

However, if such a system for blowing in filtered air prevents the introduction of pathogenic agents into the facility, the system does not act on the pathogenic agents that can be present in the first zone that delimits the first enclosure and inside of which the blow-molding unit and the transfer device are arranged.

The use of sterilizing solutions such as sodium hydroxide (NaOH) or hydrogen peroxide (H₂O₂) for carrying out decontamination in the first zone in particular runs into risks of corrosion of elements made in materials other than stainless steel, in particular for the molds of the blow-molding unit.

Consequently, the transfer device that is arranged inside the first enclosure with the blow-molding unit is no longer in parallel the object of particular action whose aim is to destroy the pathogenic agents that can be present on at least one wheel for transfer of the containers.

However, in the production mode, the containers are in contact with the holding means as well as with the associated guide means of such a transfer wheel in such a way that contamination of the containers may then occur.

Contamination of a container during transfer is a particularly critical risk because a contaminated container that is transferred toward the filling unit then constitutes a carrier for introducing pathogenic agents.

In addition, and independently of the containers, such pathogenic agents that are present on a transfer wheel may also be airborne up to the second zone in which the filling unit is arranged.

These risks of contamination associated with the transfer wheel may affect all of the different actions implemented for eliminating the pathogenic agents, such as, for example, the decontamination of the filling unit.

The object of this invention is in particular to propose a facility for producing containers in which the risks of contamination of the containers are smaller and smaller, and even eliminated, and very particularly the risks of contamination that are associated with the presence of pathogenic agents in the area of the transfer device.

For this purpose, the invention proposes a facility of the type described above, characterized in that the facility comprises a device for disinfecting the transfer wheel that is controlled from outside the facility for disinfecting, by spraying at least one disinfecting agent, at least the upper part for transfer of the containers from the wheel when, in the decontamination mode, said upper part for transfer of the containers from the wheel occupies the retracted position.

Thanks to the disinfecting device according to the invention, said at least one transfer wheel is disinfected, eliminating the pathogenic agents that could be present there.

Advantageously, such an operation for disinfecting the transfer wheel is carried out while the facility operates in the decontamination mode.

Preferably, the disinfecting operation is implemented before any containers are put into production so as to reduce, and even to eliminate, the risks of subsequent contamination of the filling unit, in particular contamination of a container by contact between the container and the transfer wheel during production.

According to other characteristics of the invention:
The disinfecting device consists of at least one gun arranged inside the facility and able to be operated by an operator from outside the facility, by means of remote handling means, for spraying said at least one disinfecting agent onto at least said upper part for transfer of the containers from the wheel;
The disinfecting device consists of at least one automatic disinfecting module that, controlled from outside the facility, comprises means for spraying said at least one disinfecting agent for disinfecting at least said upper part for transfer of the containers from the wheel;
Said automatic disinfecting module is mounted to move between at least:
A disinfection position, occupied in the decontamination mode, in which said spraying means are able to disinfect at least the upper part for transfer of the containers from the wheel; and
A rest position, occupied in the production mode, in which said disinfecting module is retracted;
The automatic disinfecting module comprises at least one arm of said spraying means of the disinfecting agent and said arm is moved selectively by at least one actuator to be deployed toward the disinfection position or retracted toward the rest position;
Said means for spraying the disinfecting agent are mounted to move in rotation around an axis, respectively between at least a first angular position and a second angular position, to carry out, in said disinfection position, a sweeping action of at least said upper part for transfer of the containers from the wheel;
Said upper part for transfer of the containers from the wheel comprises at least holding means with which are associated guide means that, in the production mode, work respectively with the transfer of the containers, and the automatic disinfecting module comprises at least first spraying means for disinfecting said holding means and second spraying means for disinfecting said guide means;
Said at least one disinfecting agent is formed completely or in part by a compound of the alcohol family that is sprayed in the liquid state by the disinfecting device to disinfect at least said upper part for transfer of the containers from the wheel;
At least one part of the disinfecting device or the upper part for transfer of the containers from the wheel occupying said retracted position is able to carry out one relative movement in relation to the next to perfect the application of said at least one disinfecting agent;
Said drive system of the transfer wheel is able to drive in rotation at least said upper part for transfer of the containers from the wheel when, in the decontamination mode, said upper part for transfer of the containers occupies said retracted position, owing to which at least said upper part for transfer of the containers from the wheel is driven by a relative movement in relation to the disinfecting device;
Said drive system of the wheel for transfer of the containers comprises at least one primary shaft made in at least two parts, respectively a first upper part to which is connected at least said upper part for transfer of the containers, and a second lower drive part, with said first and second parts being linked in rotation to a secondary shaft;
The first and second parts of the primary shaft are respectively linked in rotation to the secondary shaft by mating shapes; preferably, said connection in rotation is made by engagement;
A secondary clutch mechanism is associated with the secondary shaft to control selectively its driving by motorized means;
Said secondary clutch mechanism occupies at least:
   An engaged state when said at least one upper part for transfer of the containers from the wheel is in the retracted position for coupling in rotation, by means of the secondary shaft, said upper part of said primary shaft to said motorized means so as to drive in rotation said upper part for transfer of the containers, and
   A disengaged state when said at least one upper part for transfer of the containers from the wheel is in the transfer position for disconnecting said secondary shaft from said motorized means.

Other characteristics and advantages of the invention will emerge from reading the following description for the understanding of which reference will be made to the accompanying drawings in which:

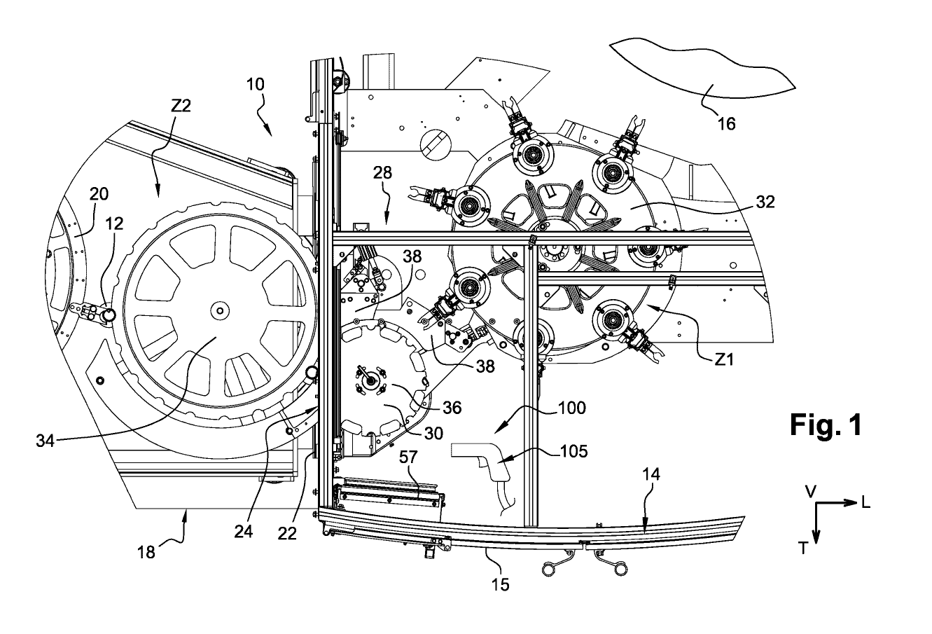

FIG. 1 is a top view that partially shows an embodiment of a facility for producing containers and that illustrates more particularly the common junction part between the enclosures of the blow-molding unit and the filling unit in which an opening is created for the transfer of containers;

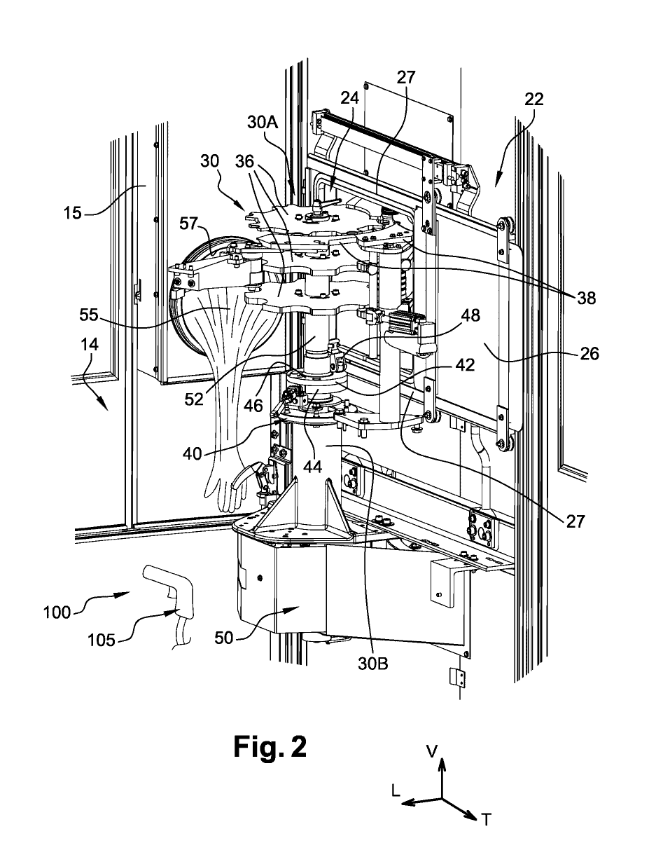

FIG. 2 is a three-quarter rear view that shows the facility according to FIG. 1 and that illustrates the transfer wheel of the transfer device that, adjacent to the opening, is in the transfer position;

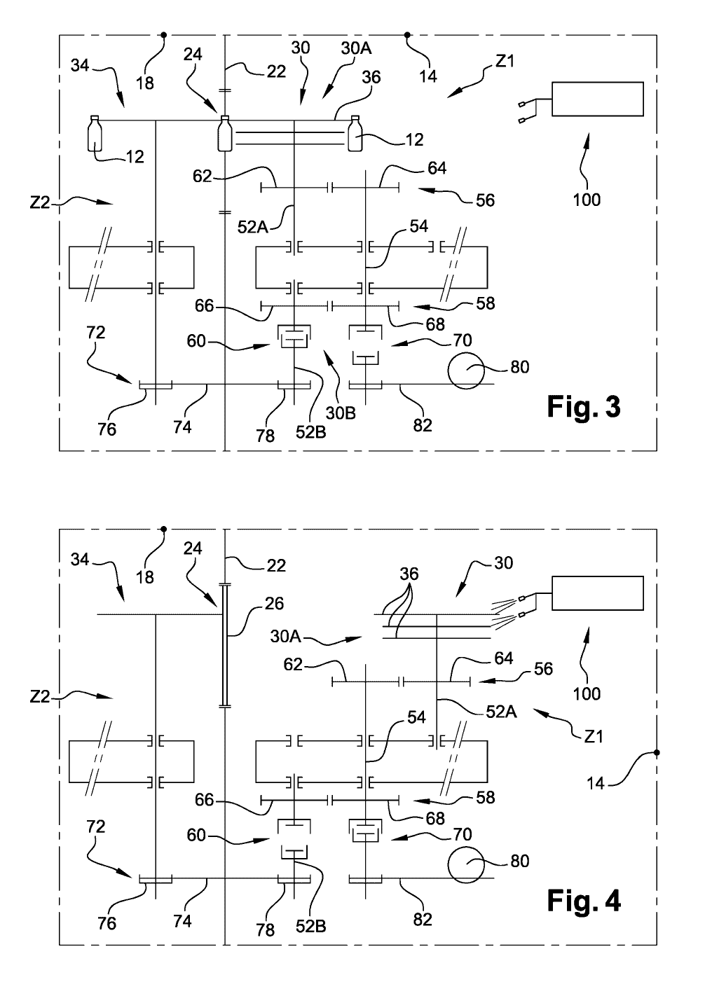

FIGS. 3 and 4 are diagrammatic representations of the drive system of the transfer wheel, which representations illustrate said system respectively in the transfer and disinfection positions in which at least the upper part for transfer of the containers from the transfer wheel is able to be driven in rotation;

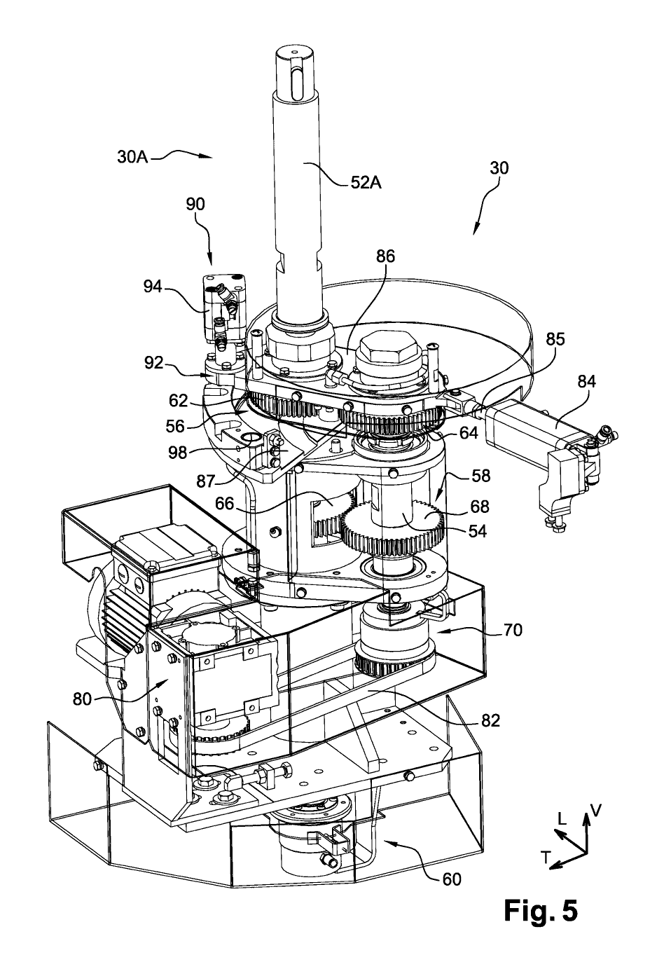

FIGS. 5 to 7 are perspective views that show in detail a transfer wheel that comprises a drive system according to FIGS. 3 and 4 and that illustrate the upper part of the transfer wheel in the transfer position and in the retracted position;

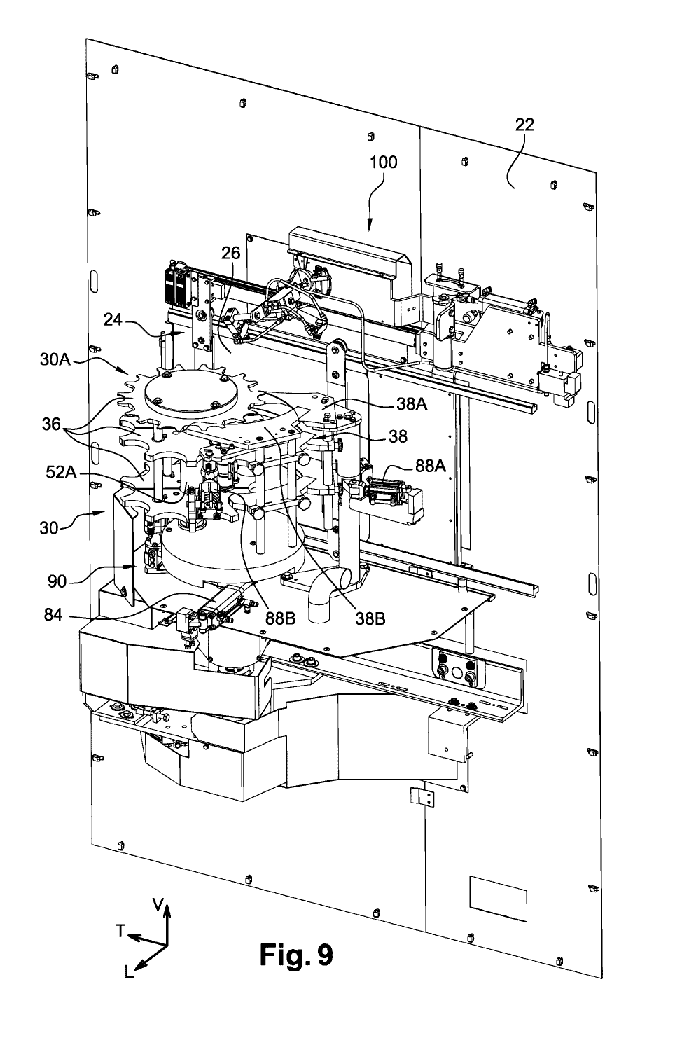

FIGS. 8 and 9 are three-quarter perspective views that partially show the facility and more particularly the transfer wheel according to FIGS. 5 to 7 that is adjacent to the common part of the first and second enclosures comprising the opening and that respectively illustrate the facility in the transfer mode and in the decontamination mode, with the upper part of the transfer wheel respectively occupying the transfer position and the retracted position while the disinfecting device occupies respectively the rest position and the disinfection position;

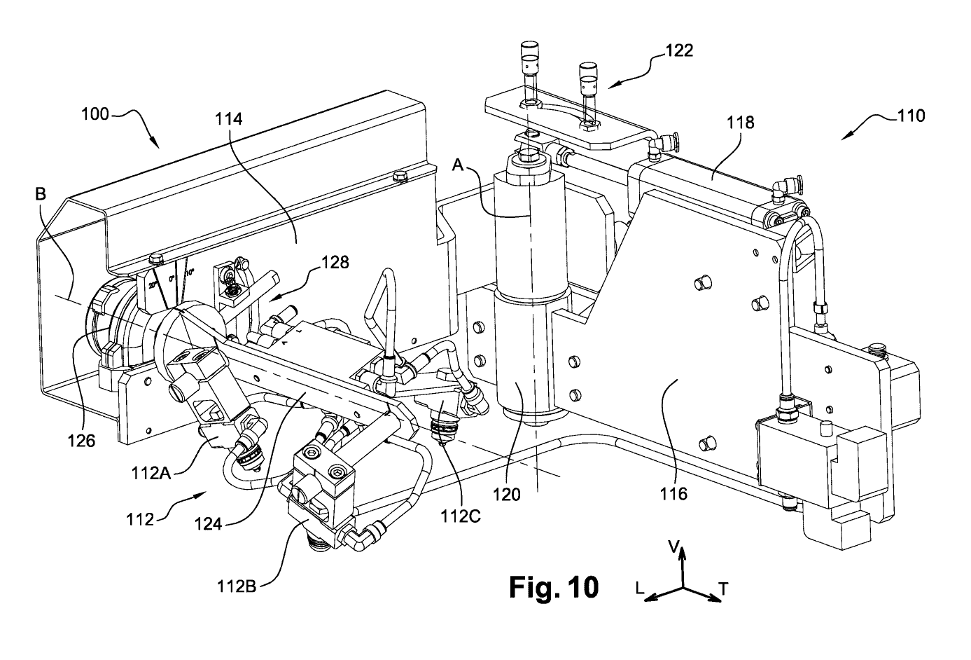

FIG. 10 is an entirely automated perspective view that shows a disinfecting device, according to the second embodiment, of the facility;

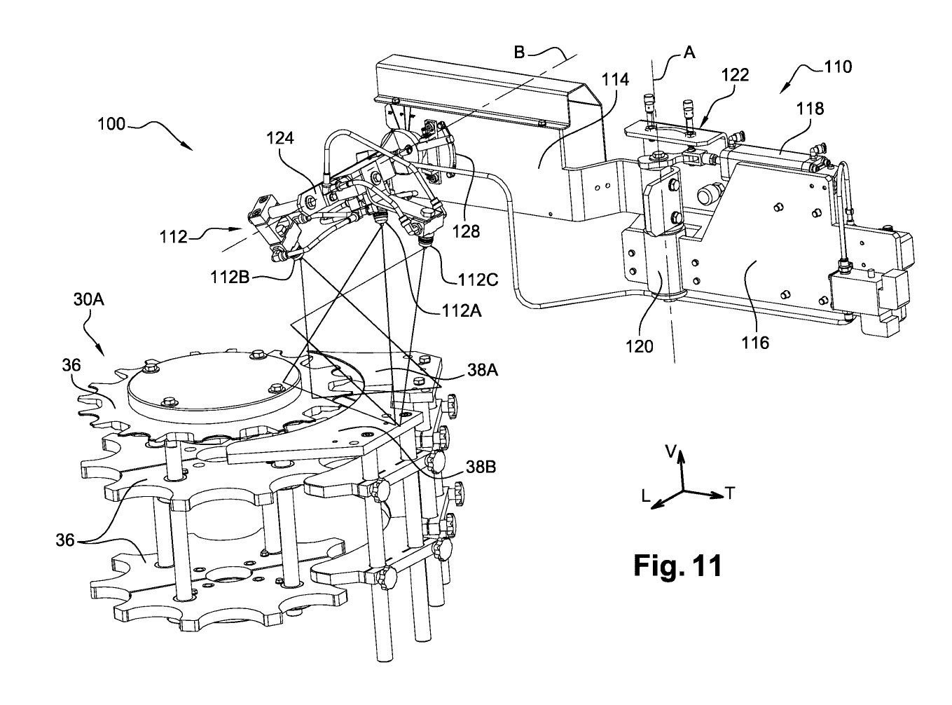

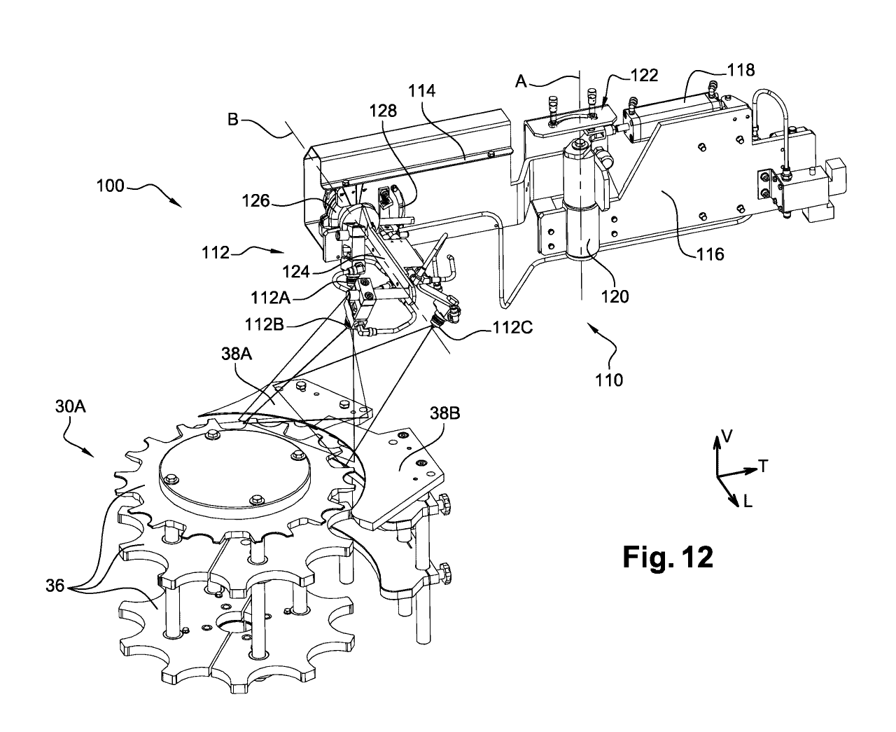

FIGS. 11 and 12 are perspective views that show the disinfecting device according to FIG. 10 and that illustrate the spraying of the disinfecting agent by the nozzles in the first angular position and in the second angular position.

In the description below, similar or identical elements will be designated by the same references.

In the description, the following will be used in a non-limiting manner: the expressions such as “upstream” and “downstream,” “upper” and “lower,” “inside” and “outside,” “front” and “rear,” and the longitudinal, vertical and transverse orientations with reference to the trihedrons (L, V, T) shown in the figures and to the definitions provided in the description.

FIG. 1 shows an embodiment of a facility 10 for producing containers 12, in particular, but not exclusively, bottles.

A first embodiment of a facility 10 for producing containers 12 that is analogous to the one described in the document WO-2010/081759, whose referenced FIGS. 3 and 5 correspond to these FIGS. 1 and 2, will be described below.

Reference will therefore advantageously be made to this document for a more detailed description of the entire facility 10.

As partially shown in FIG. 1, the facility 10 comprises at least one first protective enclosure 14 that delimits a first zone Z1 inside of which is arranged at least one unit 16 for blow molding containers 12.

Preferably, the blow-molding unit 16 is able to manufacture containers 12 by blow molding or by stretch blow molding starting from preforms (not shown) that are thermally conditioned in advance in a furnace (not shown), in particular by heating by means of infrared radiation lamps.

In a known manner, such a blow-molding unit 16 comprises, upstream, a heating furnace for the thermal conditioning of the preforms, as well as transfer means (not shown) arranged at the outlet that are able to bring each of said heated preforms into one of the molds (not shown).

The blow-molding unit 16 comprises a carrousel (not shown) on the radial periphery of which said molds are circumferentially arranged, in which molds the heated preforms are transformed into a container 12 by blow molding or else by stretch blow molding according to the applications.

The facility 10 comprises a second confinement enclosure 18 that delimits a second sterile zone Z2, inside of which at least one unit 20 for filling containers 12 manufactured by the blow-molding unit 16 is arranged.

The second confinement enclosure 18 is at least in part attached to the first adjacent enclosure 14 by a common part 22.

Each of the enclosures 14 and 18 consists of a set of vertical walls that, in particular assembled with one another to form a parallelepiped as a whole, are respectively closed above and below, for example by a wall that forms a ceiling and by the floor respectively.

Preferably, at least one of the vertical walls of the enclosures 14 and 18 comprises doors so as to make possible their clearing, in particular by an operator, and access into the facility 10.

The first enclosure 14 comprises, for example, at least two access doors 15 forming a part of its front vertical wall.

The facility 10 comprises at least one opening 24 that is made in said common part 22 of the first and second enclosures 14 and 18. The opening 24 that is visible in FIG. 2 is designed to allow the transfer of the containers 12 from the blow-molding unit 16 to the filling unit 20.

Advantageously, the facility 10 comprises means 26 for sealing said opening 24 that are able to be moved selectively between at least one open position and one closed position.

The open position corresponds to the position in which the sealing means 26 allow the transfer of the containers 12 through the opening 24 and is occupied when the facility 10 is in an operating mode, a so-called production mode.

The closed position corresponds to the position in which the sealing means 26 prevent any transfer by insulating the second enclosure 18.

The closed position is in particular designed to make it possible to initiate decontamination operations of the filling unit 20 at least in the second enclosure 18, when the facility 10 is in another operating mode, a so-called decontamination mode.

Preferably, the facility 10 comprises actuating means designed to control selectively the movement of the sealing means 26 from the opening 24 to make said sealing means 26 slide between the open position corresponding to the production mode and the closed position corresponding to the decontamination mode.

Advantageously, the sealing means 26 ensure, in said closed position, a hermetic sealing of the opening 24 that is suitable for insulating the second enclosure 18 comprising the unit 20 for filling the first enclosure 14 comprising the blow-molding unit 16 of the containers 12.

The means 26 for sealing the opening 24 consist of a flap that is mounted to slide between said open and closed positions by means of two slides 27, respectively an upper slide and a lower slide.

The slides 27 of the flap 26 are integral with the common part 22 and are arranged respectively above and below the opening 24 that has here an overall rectangular shape that can be seen more particularly in FIG. 2.

The operations for decontamination of the filling unit 20 are carried out by chemical means by spraying sterilizing solutions, such as sodium hydroxide (NaOH) or hydrogen peroxide (H₂O₂), inside the second enclosure 18.

The spraying of such sterilizing solutions for decontaminating the second enclosure 18 is in particular made possible by the use of stainless steel or other compatible materials for producing the parts such as those of the filling unit 20.

The facility 10 comprises a transfer device 28 for ensuring the transfer of the containers 12 between the blow-molding unit 16 and the filling unit 20.

The transfer device 28 comprises at least one transfer wheel 30 that is adjacent to the opening 24 made in the common part 22 and that is designed to transfer, in the production mode, the containers 12 through said opening 24.

Preferably, in addition to the transfer wheel 30, the transfer device 28 comprises two other transfer wheels 32 and 34 that are respectively arranged upstream and downstream from the wheel 30.

The different wheels 30, 32 and 34 of the transfer device 28 are arranged in relation to one another in such a way as to each have an arc portion tangent to the portion of the adjacent wheel so as to determine a zone in which the transfer of the containers 12 from one to the next is performed.

The first transfer wheel 32 is arranged between the inlet of the transfer wheel 30 and an exit zone of the carrousel of the blow-molding unit 16 in which the molds are controlled by opening to allow the extraction of the manufactured containers 12 owing to associated blow-molding or stretch-blow-molding means.

The first transfer wheel 32 is designed to ensure the extraction of the manufactured containers 12 from the molds and their transfer downstream, for example by means of transfer arms equipped at their free ends with gripping means, such as clamps.

The second transfer wheel 34 is arranged, downstream, between the outlet of the transfer wheel 30 and another carrousel that comprises the filling unit 20, with the filling stations of the containers 12 being distributed circumferentially in a uniform manner around said carrousel.

Advantageously, the facility 10 is designed in such a way as to reduce or eliminate the different risks of contamination, in particular and according to the teachings delivered by the document WO-2010/081759, those linked to the intervention by an operator to act on the transfer wheel 30 for the purpose of allowing the opening or the closing of the opening 24.

The transfer wheel 30 comprises at least one part that is respectively mounted to move between at least one first transfer position and a second retracted position.

The first transfer position is occupied when the facility 10 is in the production mode, and the second retracted position is occupied when the facility 10 is in the decontamination mode.

Preferably, the transfer wheel 30 comprises an upper part 30A for transferring the containers and a lower part 30B for driving the wheel 30.

The upper part 30A comprises at least holding means 36 that extend in part through the opening 24 and, in the production mode, work with the containers 12 for transferring them.

The holding means 36 consist of, for example, three superposed plates that are each provided circumferentially with slots, each slot being designed to work with a part of the container.

The upper plate works with the neck of the container, generally supporting it by means of a radial collar with which the container is provided, while the intermediate and lower plates work with the body of the container, locking it laterally to keep it vertical during the transfer.

Advantageously, the transfer wheel 30 comprises guide means 38 that, associated with the holding means 36, act to ensure the transfer of the containers 12.

As described in the document WO-2010/081759, said at least one movable part of the transfer wheel 30 preferably consists of the upper transfer part 30A of the containers while the lower drive part 30B of the wheel 30 is a stationary part.

The upper part 30A of the transfer wheel 30 is mounted to move between at least:
The first transfer position in which at least said upper part 30A of the wheel 30 extends through the opening 24 to ensure the transfer of the containers 12 during operation in the production mode of the facility 10, and
The second retracted position in which at least said upper part 30A of the wheel 30 is moved to allow the closing of the opening 24 by the associated sealing means 26 for the purpose of the operation of the facility 10 in the decontamination mode.

In this first embodiment, the transfer wheel 30 of the facility 10 is preferably of a design analogous to the one described in the document WO-2010/081759.

The transfer wheel 30 comprises at least one flange 40 that comprises an upper element 42 and a lower element 44 that are respectively integral with the upper part 30A and the lower part 30B.

Articulation means 46, such as a pivot, are arranged between the upper element 42 and the lower element 44 of the flange 40 for making possible the movement of the upper part 30A in relation to the lower part 30B.

Preferably, the transfer wheel 30 comprises locking means 48 that can immobilize the movable upper part 30A of the wheel 30 in at least one of said transfer or retracted positions.

The facility 10 comprises a drive system 50 of the transfer wheel 30 to ensure the driving in rotation of at least the upper part 30A for transfer of the containers 12 from the wheel 30 when, in the production mode, said upper part 30A occupies the transfer position.

In the document WO-2010/081759, said drive system 50 of the transfer wheel 30 comprises a drive shaft that comprises said flange 40 placed at the junction of a first shaft segment integral with the upper flange element 42 and a second shaft segment integral with the lower flange element 44.

The holding means 36 are in particular mounted on the first shaft segment to form said upper part 30A for transfer of the containers from the wheel 30.

The second shaft segment is connected to motorized means that advantageously consist of the means for driving in rotation the filling unit 20 and/or the second transfer wheel 34 so as to ensure, in the production mode, a synchronous driving in the direction of transfer of the containers 12, from the upstream blow-molding unit 16 toward the downstream filling unit 20.

The means for driving the filling unit 20 in rotation comprise at least motorized means and transmission means, such as a belt, which ensure the transmission of the torque between said motorized means and the shaft of the transfer wheel 30.

The drive system 50 comprises a clutch mechanism that is associated with the drive shaft for selectively coupling said shaft in rotation and driving it in rotation only in the production mode when the upper part 30A occupies its transfer position.

When the upper part 30A occupies its retracted position in the decontamination mode, the clutch mechanism is then opened to interrupt the driving in rotation of the upper part 30A.

According to the teachings of the document WO-2010/081759, the movement of the upper part 30A of the transfer wheel 30 between said transfer and retracted positions is carried out, manually or automatically, from outside the facility 10, i.e., from outside the first enclosure 14 to the inside of which is arranged said transfer wheel 30.

As a variant, the entire transfer wheel 30 is mounted to move, for example by being mounted on a carriage that can allow its movement between said transfer and retracted positions and always from outside the facility.

In such a variant, the transfer wheel 30 advantageously comprises its own means for driving in rotation, independent of those of the filling unit 20.

Advantageously, the physical presence of an operator inside the facility 10 and most particularly in the first zone Z1 of the first enclosure 14 is therefore no longer necessary, owing to which the associated risks of contamination are totally eliminated.

As explained in the preamble, the object of this invention is to reduce the microbiological risks within a facility 10 for producing containers by combating the different risks of contamination of the containers by pathogenic agents, and this is done to continue to improve the food safety of the containers.

For this purpose, the invention proposes carrying out an operation for disinfecting at least one part of the transfer wheel 30 of the production facility 10.

Advantageously, the disinfecting operation is carried out when the facility 10 is in the decontamination mode, for example parallel to the implementation of the decontamination operation of the filling unit 20.

Advantageously, the disinfecting of the transfer wheel 30 is performed in the background without affecting the production of the containers that succeeds or precedes the implementation of said operations for decontamination of the unit 20 for filling and disinfecting at least a part of the transfer wheel 30.

The use of the term “disinfection” is in particular designed to aid in the distinction between the new operation implemented for treating the transfer wheel 30 and that of “decontamination,” carried out for treating the filling unit 20.

It is to be understood, however, that disinfecting, like decontamination, strives toward one and the same goal, i.e., the destruction of pathogenic agents (germs, spores, bacteria, etc.) that may be present on the treated surfaces, in particular but not exclusively surfaces designed to be in contact with the containers.

The quantity and the type of pathogenic agents that are destroyed are in particular based on products that are used during said operations for decontamination and disinfecting.

Recall that the quantity of pathogenic agents may be identified by counting after in particular operations of washing, filtering, and cultivation.

A logarithmic reduction of the number of pathogenic agents, for example said to be on the order of 3 Log (or else 3D) equivalent to 1,000 units (10³), is thus determined.

By way of example, the operation of decontamination by chemical means of the filling unit 20 with sterilizing solutions such as sodium hydroxide (NaOH) or hydrogen peroxide (H₂O₂) makes it possible to obtain results that can range up to 6 Log.

Such sterilizing solutions may also be used for the operation of disinfecting at least one part of the transfer wheel, in particular when the treated surfaces are made of appropriate materials, such as stainless steel.

However, independently of the risks of corrosion linked to the use of sterilizing solutions such as sodium hydroxide (NaOH) or hydrogen peroxide (H₂O₂), it is also advisable to take into consideration the subsequent elimination of said sterilizing solutions at the end of the disinfecting operation.

Actually, the regulation in the agricultural field generally imposes requirements as to the residual presence of certain products, such as the presence of traces of sterilizing solution, in or on the containers.

Advantageously, the disinfecting operation according to the invention is carried out with at least one disinfecting agent that is formed completely or in part by a compound of the alcohol family.

Preferably, said at least one disinfecting agent comprises ethanol, for example ethanol diluted to 70%.

Preferably, said at least one disinfecting agent is applied to the liquid state, in particular by spraying.

Advantageously, said at least one disinfecting agent is then eliminated naturally by evaporation.

As a variant, means such as hot air are used to force evaporation and to reduce the time that is necessary for the elimination of the disinfecting agent.

As a variant, said at least one disinfecting agent could be applied to the gaseous state.

For carrying out said disinfecting operation according to the invention, the facility 10 comprises at least one disinfecting device 100 that is arranged inside the first protective enclosure 14, delimiting the first zone Z1 of the facility 10.

Advantageously, said at least one disinfecting device 100 is controlled from outside the facility 10.

The disinfecting device 100 is designed to disinfect at least a part of the transfer wheel 30, in particular the surfaces that are in contact with the containers, during the transfer that is performed, in the production mode, from the blow-molding unit 16 to the filling unit 20.

The device 100 for disinfecting the transfer wheel 30 is controlled manually or automatically from outside the facility 10 for disinfecting, by spraying at least one disinfecting agent, at least the upper part 30A for transfer of the containers 12 from the wheel 30.

The spraying of said at least disinfecting agent is carried out in the decontamination mode of the facility 10, when said upper part 30A for transfer of the containers 12 from the wheel 30 occupies the retracted position.

In this first embodiment, the disinfecting device 100 comprises spraying means that consist of at least one gun 105 that can spray said at least one disinfecting agent on at least said upper part 30A for transfer of the containers 12 from the wheel 30.

Preferably, the gun 105 is fed with disinfecting agent by a reservoir. The disinfecting agent in the liquid state is, for example, sprayed by means of a gas, such as compressed air.

Preferably, the gun 105 is arranged close to the surfaces of the transfer wheel 30 that is to be disinfected and inside the first enclosure 14, in the transfer part of the first zone Z1.

Advantageously, the gun 105 is able to be operated by an operator by means of remote handling means making it possible to carry out said disinfecting operation from outside the facility 10.

Like the retraction of the upper part 30A for transfer of the containers from the transfer wheel 30, the disinfecting operation is carried out without an operator being inside the facility 10 and without clearing the doors 15 to enter in particular the part of the first zone Z1 where the transfer wheel 30 and said gun 105 forming the disinfecting device 100 are located.

As shown in FIG. 2, said remote handling means are formed by at least one glove 55 extended by a cuff and connected in an airtight manner to an opening 57 made in the first protective enclosure 14.

Advantageously, the risks of contamination linked to the presence of an operator inside the first zone Z1 are totally eliminated, with the intervention by the operator being performed by means of the glove 55 through the first enclosure 14 but never without the latter being physically in contact with the air that is present in the first zone Z1.

The operator therefore cannot create a carrier for introducing pathogenic agents that may subsequently affect the containers 12 during operation in the production mode.

By means of the glove 55, the operator may operate the gun 105 and spray the disinfecting agent on the different surfaces to be disinfected.

The handling glove 55 makes it possible for the operator to carry out manually both the retraction of the upper part 30A of the transfer wheel 30 (when the former is not automated) and the disinfecting operation by means of said gun 105.

As a variant, the disinfecting operation is carried out automatically and not manually, for example by means of at least one gun 105 moved automatically into the space following one or more axes by controlled handling means, such as a robotic arm controlled from outside the facility 10.

The disinfecting operation is in particular carried out on at least the holding means 36 of the upper part 30A of the transfer wheel 30 and the associated guide means 38.

Advantageously, a disinfecting device 100 such as the gun 105 may easily be introduced into an existing facility 10 such as the one described and shown in the document WO-2010/081759, without requiring significant modifications.

Although the implementation of the disinfecting operation according to the first embodiment that was just described can be fully satisfactory, improvements can, however, also be provided.

First of all, the transfer wheel 30 remains stationary during the disinfecting operation; the only relative movement between the gun 105 forming the disinfecting device 100 and the transfer wheel 30 in the case of a manual embodiment corresponds to the movability offered by the hand of the operator in combination with the glove 55.

Actually, when the transfer wheel 30 is made in two parts, respectively an upper transfer part 30A and a lower drive part 30B, the first upper part 30A itself is moved from the transfer position to the retracted position, and in the decontamination mode, the driving is interrupted in particular for allowing retraction.

It will be understood that accessibility like visibility are less for the operator on the surfaces that are further away, such as those of the guide means 38, than on the surfaces that, in the immediate vicinity, face it directly.

Consequently, the quality of the results of the disinfection may be affected by a more or less homogeneous and total application of the disinfecting agent when the disinfection is carried out manually by an operator according to the first embodiment that was just described.

The fact that the disinfecting operation is carried out manually by an operator operating the gun 105 also poses the question of the reliability linked to the human factor.

It will be understood that it is not possible to ensure completely perfect repeatability of gestures for the same operator, or between two successive operators, independently of any application that each could nevertheless perform.

There is therefore a problem of reliability leading to a search for ways to remove the human factor linked to any intervention by an operator.

By eliminating the manual intervention by an operator during operation, the main object in mind is not so much to reduce the costs of using such a facility 10 but rather to have the assurance of perfect repeatability and therefore reliability in the results of the disinfecting operation.

Below, a second embodiment of a disinfecting device 100 for a facility 10 for producing containers will be described.

According to a first characteristic of the second embodiment, at least the upper part 30A of the transfer wheel 30 may be driven in rotation when, in the decontamination mode, at least said part 30A occupies the retracted position.

By comparison, recall that in the facility according to the document WO-2010/081759, the upper part for transfer of the containers from the wheel 30 was not driven in rotation in the retracted position.

According to a second characteristic of this second embodiment, the disinfecting operation is carried out entirely in an automated manner.

By comparison with the first embodiment, any manual intervention by an operator is consequently eliminated, owing to which the above-mentioned risks relative to reliability, repeatability of the result of the disinfecting operation, etc., linked to the human factor are also eliminated.

Of course, the first and second characteristics are advantageously combined but are independent of one another.

The driving in rotation of the upper part of the transfer wheel 30 in the retracted position may also be implemented with another disinfecting device, in particular with a gun 105 according to the first embodiment.

Below, a new design of a transfer wheel 30 comprising in particular an upper part 30A for transfer of the containers 12 that is able to be driven in rotation will be described, while said part 30A occupies the retracted position.

Advantageously, at least said upper part 30A for transfer of the containers 12 from the wheel 30 occupying said retracted position is able to carry out a relative movement in relation to the disinfecting device 100 for perfecting the application of said at least one disinfecting agent during the disinfecting operation.

Preferably, at least one part of the disinfecting device 100 and said upper part 30A for transfer of the containers 12 from the wheel 30 may respectively carry out one relative movement in relation to the next.

In this second embodiment, the drive system 50 of the transfer wheel 30 is able to drive in rotation at least said upper part 30A for transfer of the containers 12 comprising holding means 36 when, in the decontamination mode, said upper part 30A for transfer of the containers 12 occupies said retracted position.

By comparison with the first embodiment in which the disinfecting device 100 formed by the gun 105 was able to be driven by a relative movement in relation to the transfer wheel 30, it is—in this second embodiment—at least said upper part 30A for transfer of the containers 12 from the wheel 30 that is driven by a relative movement in relation to the disinfecting device 100.

FIGS. 3 and 4 show diagrammatically a transfer wheel 30, respectively in the transfer position that is occupied when the facility 10 operates in the production mode of the containers 12 and in the retracted position that is occupied when the facility 10 operates in the decontamination mode.

As illustrated in FIG. 3, the transfer wheel 30 comprises at least holding means 36 for transferring the containers 12 through the opening 24 made in the common part 22 of the enclosures 14 and 18.

The holding means 36 of the containers 12 comprise three superposed plates, as described above in the first embodiment. Preferably, at least the upper plate working with the neck of the container is made of stainless steel, such as 316L stainless steel.

Preferably, the transfer wheel 30 comprises guide means 38 that act in a complementary manner to said holding means 36 during the transfer of the containers 12 but that are not shown in FIGS. 3 and 4.

The drive system 50 of the transfer wheel 30 of the containers 12 comprises at least one primary shaft 52 made in at least two parts, respectively a first upper drive part 52A and a second lower drive part 52B, with said first and second parts 52A and 52B being linked in rotation to a secondary shaft 54.

The first upper part 52A of the primary shaft 52 supports the holding means 36 with which said first upper part 52A constitutes said upper part 30A for transfer of the containers 12 from the wheel 30.

The second lower drive part 52B is connected by means of the drive system 50 that acts in the production mode for entraining, by means of the secondary shaft 54, said upper part 30A for transfer of the containers 12 from the wheel 30.

The first and second parts 52A and 52B of the primary shaft 52 are respectively linked in rotation to the secondary shaft 54 by mating shapes.

Preferably, the connection in rotation of the primary shaft 52 and the secondary shaft 54 is done by means of engaging means.

The engaging means comprise at least the first engaging means 56 that act for linking in rotation the secondary shaft 54 with said first upper engaging part 52A and second engaging means 58 that act for linking in rotation the secondary shaft 54 with said second lower part 52B of the primary shaft 52.

Advantageously, the first engaging means 56 allow a swinging movement of the first upper part 52A of the primary shaft 52 in relation to the secondary shaft 54 so as to move at least said upper part 30A for transfer of the containers 12 between said transfer and retracted positions.

As FIGS. 3 and 4 illustrate by comparison, the upper part 30A for transfer of the containers carries out, between said transfer and retracted positions, a circular movement around the secondary shaft 54.

This swinging movement corresponding to the retraction is made possible by the division into two parts of the primary shaft 52 and by the first engaging means 56.

The drive system 50 of the transfer wheel 30 comprises at least one primary clutch mechanism 60.

Preferably, the primary clutch mechanism 60 is a pneumatic-type mechanism.

Advantageously, the primary clutch mechanism 60 associated with said second lower drive part 52B of the primary shaft 52 comprises a torque limiter.

Preferably, the first engaging means 56 comprise at least one gearwheel 62 that is integral in rotation with the first upper part 52A of the primary shaft 52 and a gearwheel 64 that is integral in rotation with the upper segment of the secondary shaft 54.

The wheel 62 and the wheel 64 forming the first engaging means 56 each comprise an outer set of teeth, and said sets of teeth work together to ensure a transmission of the torque between the primary shaft 52 and the secondary shaft 54.

Advantageously, the first engaging means 56 make it possible to carry out the swinging movement corresponding to the retraction, while maintaining the possibility for the upper part 30A of the transfer wheel 30 to be driven in rotation and in particular in the retracted position.

Preferably, the second engaging means 58 comprise at least one gearwheel 66 that is integral in rotation with the second lower part 52B of the primary shaft 52 and a gearwheel 68 that is integral in rotation with the lower segment of the secondary shaft 54.

Like the first engaging means 56, the wheel 66 and the wheel 68 forming the second engaging means 58 each comprise an outer set of teeth that work together for ensuring a transmission of the torque between the primary shaft 52 and the secondary shaft 54.

The primary clutch mechanism 60 is associated with said second lower drive part 52B of the primary shaft 52 to control its driving selectively, in particular by motorized means 72.

As indicated above for the first embodiment, the motorized means 72 advantageously consist of the means for driving in rotation the filling unit 20 and/or the second transfer wheel 34 of the transfer device 28 of the facility 10.

Preferably, the motorized means 72 are linked to the primary shaft 52 by movement transmission means 74, such as a belt that works with two drive pulleys 76 and 78 respectively linked in rotation, one 76 to the motorized means 72 forming a driving element and the other 78 to the primary shaft 52 forming a driven element.

The primary clutch mechanism 60 may occupy at least one engaged state and one disengaged state for transmitting the torque selectively when the upper part 30A for transfer from the wheel 30 is in the transfer position, with the facility 10 operating in the production mode.

The primary clutch mechanism 60 occupies an engaged state for coupling in rotation said lower part 52B of said primary shaft 52 to said motorized means 72 when said at least one upper part 30A for transfer of the containers 12 from the wheel 30 is in the transfer position.

The primary clutch mechanism 60 occupies a disengaged state for disconnecting said lower part 52B from said primary shaft of said motorized means 72 when said at least one upper part 30A for transfer of the containers 12 from the wheel 30 is in the retracted position.

The primary clutch mechanism 60 is respectively in the engaged state in FIG. 3 and in the disengaged state in FIG. 4.

The transfer wheel 30 comprises a secondary clutch mechanism 70 that is associated with the secondary shaft 54 for selectively controlling its driving by secondary motorized means 80, such as a motor.

The secondary motorized means 80 are linked in rotation, for example, to the secondary shaft 54 by means of secondary transmission means 82.

The secondary clutch mechanism 70 may occupy at least an engaged state and a disengaged state for transmitting the torque selectively when the upper part 30A for transfer from the wheel 30 is in the retracted position, with the facility 10 operating in the decontamination mode.

The secondary clutch mechanism 70 occupies said engaged state, illustrated in FIG. 4, when said at least one upper part 30A for transfer of the containers 12 from the wheel 30 is in the retracted position and this is done for coupling in rotation, by means of the secondary shaft 54, said upper part 52A of said primary shaft 52 to said secondary motorized means 80 so as to drive in rotation said upper part 30A for transfer of the containers 12.

The secondary clutch mechanism 70 occupies said disengaged state, illustrated in FIG. 3, when said at least one upper part 30A for transfer of the containers 12 from the wheel 30 is in the transfer position and this is done for disconnecting said secondary shaft 54 from said secondary motorized means 80.

As shown in FIGS. 3 and 4, the primary clutch mechanism 60 is mounted on the second lower part 52B for driving the primary shaft 52, between the second means 58 for engaging with the secondary shaft 54 and the motorized means 72.

The secondary clutch mechanism 70 is itself mounted on the secondary shaft 54 between the second means 58 for engaging with the primary shaft 52 and said secondary motorized means 80.

Advantageously, the drive system 50 made in particular of the two shafts 52 and 54 and associated engaging means 56 and 58 makes it possible, by controlling the clutch mechanisms 60 and 70, to ensure selectively the driving in rotation of the upper transfer part 30A in each of said transfer and retracted positions.

The main steps carried out in the area of the transfer wheel 30 for performing an entirely automated disinfecting operation according to the second embodiment of the invention will be described more particularly below.

FIGS. 5 to 7 show a transfer wheel 30 according to the second embodiment, advantageously combining, on the one hand, the first characteristic concerning the driving of the upper part 30A in the retracted position as described and illustrated diagrammatically in FIGS. 3 and 4, and, on the other hand, the second characteristic concerning an automation of its retraction.

To carry out selectively the driving of the upper part 30A for transfer of the containers 12 into each of said transfer and retracted positions, the primary clutch mechanism 60 and the secondary clutch mechanism 70 of the wheel 30 are controlled in the manner described below.

In the transfer position of the upper part 30A of the wheel 30 shown in FIG. 5, the torque delivered by the motorized means 72 enters via the lower part 52B of the primary shaft 52 and then, with the primary clutch mechanism 60 occupying its engaged state, is transmitted by means of the second engaging means 58 to the secondary shaft 54.

The secondary shaft 54 then transmits this torque, by means of the first engaging means 56, to the upper part 52A of the primary shaft 52 forming with the holding means 36 said upper part 30A of the transfer wheel 30.

In the production mode, the secondary clutch mechanism 70 is open, occupying its disengaged state, in such a way that the motorized means 80 then may not drive the secondary shaft 54 in rotation.

To switch from the production operating mode to the decontamination mode, the pneumatic-type primary clutch mechanism 60 is driven to open to create a change in state, from the engaged state to the disengaged state, so as to interrupt the driving in rotation by the motorized means 72 of the upper part 30A of the wheel 30 occupying its transfer position.

It will be noted that the holding means 36 have not been shown on the upper part 52A in FIGS. 5 to 7.

The primary clutch mechanism 60 and the secondary clutch mechanism 70 then respectively occupy their disengaged state and the upper part 30A of the wheel 30 is then free to be retracted from outside the facility 10.

Actually, the primary shaft 52 and the secondary shaft 54 are free in rotation, with the first engaging means 56 and the second engaging means 58 not opposing the swinging movement of the upper part 30A of the wheel 30 around an axis that corresponds to the secondary shaft 54.

By comparison with the first embodiment, the retraction of the upper part 30A for transfer of the containers 12 from the wheel 30 is advantageously automated and no longer carried out manually by an operator.

Advantageously, the transfer wheel 30 comprises actuating means 84 for automatically moving, always from outside the facility 10, said upper part 30A for transfer of the containers 12 between said transfer and retracted positions.

Preferably and as illustrated in FIGS. 5 to 7, the actuating means 84 consist of at least one jack comprising a rod 85. The rod 85 of the jack 84 is connected at its free end to a plate 86 that maneuvers the upper part 52A of the primary shaft 52 belonging to the upper part 30A for transfer of the containers 12.

The upper part 52A of the primary shaft 52 and the secondary shaft 54 vertically pass through the plate 86; the plate 86 may pivot in relation to the secondary shaft 54 and then entrains said upper part 52A.

The upper part 30A of the transfer wheel 30 comprises a support 87 that is stationary in relation to the plate 86, the upper part 52A and the gearwheel 62 that move during the rotation of said upper part 30A between the transfer position and the retracted position.

Preferably, the guide means 38 associated with the holding means 36 of the upper part 30A are mounted to move and are retracted automatically to make possible in particular the retraction of the upper part 30A of the transfer wheel 30 and the closing of the opening 24 by the sealing flap 26 in the decontamination mode.

The guide means 38 are mounted to move between a guide position and a retracted position, in particular shown in FIGS. 8 and 9 respectively.

Advantageously, said guide means 38 are moved automatically between said guide and retracted positions by means of at least one actuator, such as a jack.

In this second embodiment, the guide means 38 comprise first guide means 38A and second guide means 38B that act respectively during the transfer of the containers 12 in the production mode.

Preferably, the first guide means 38A are retracted automatically by an associated jack 88A and the second guide means 38B are retracted by an associated jack 88B.

Advantageously, all of said actuators 84, 88A and 88B are controlled from the outside, promoting the elimination of any presence of an operator inside the first zone Z1 that is delimited by the first enclosure 14, and they also make it possible to reduce the time necessary for the retraction maneuvers.

Advantageously, the transfer wheel 30 comprises automatic locking means 90 that are controlled from outside to immobilize said upper part 30A for transfer of the containers 12 into the transfer position and the retracted position respectively.

The locking means 90 of the wheel 30 comprise at least one locking element 92 and an associated actuator 94 for selectively moving said locking element 92 between:
A retracted position in which at least the upper part 30A for transfer of the containers 12 from the wheel 30 is free to be moved between said transfer and retracted positions, and
An engaged position in which said locking element 92 immobilizes in position said upper part 30A for transfer of the containers 12 from the wheel 30 or in the retracted position.

The locking means 90 are carried by the movable plate 86 that is actuated by the jack 84 and are therefore moved simultaneously with the upper part 30A.

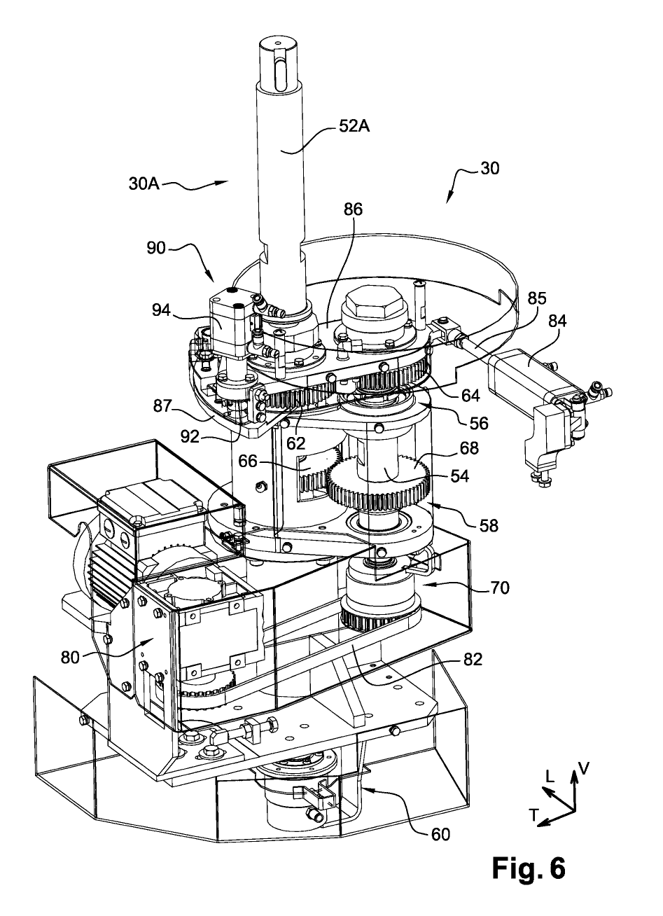

As illustrated in FIGS. 5 and 6, the locking element 92 is introduced into a first housing 96 for locking said upper part 30A in the transfer position or in a second housing 98 for locking said upper part 30A in the retracted position.

The housings 96 and 98 are carried by the support 87, which is stationary in relation to the plate 86 that, moved by the jack 84, carries the locking means 90.

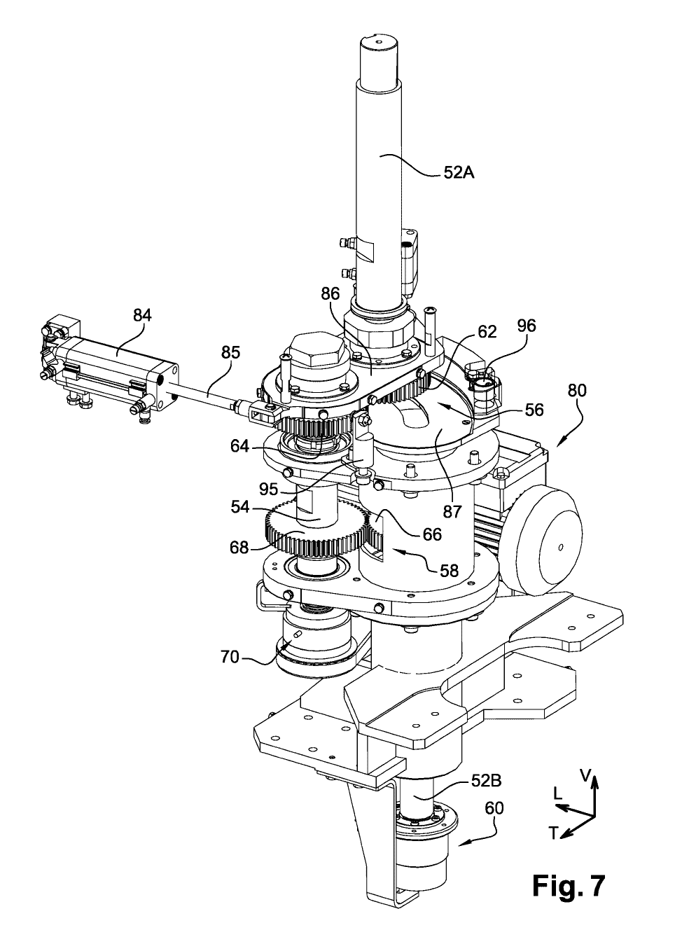

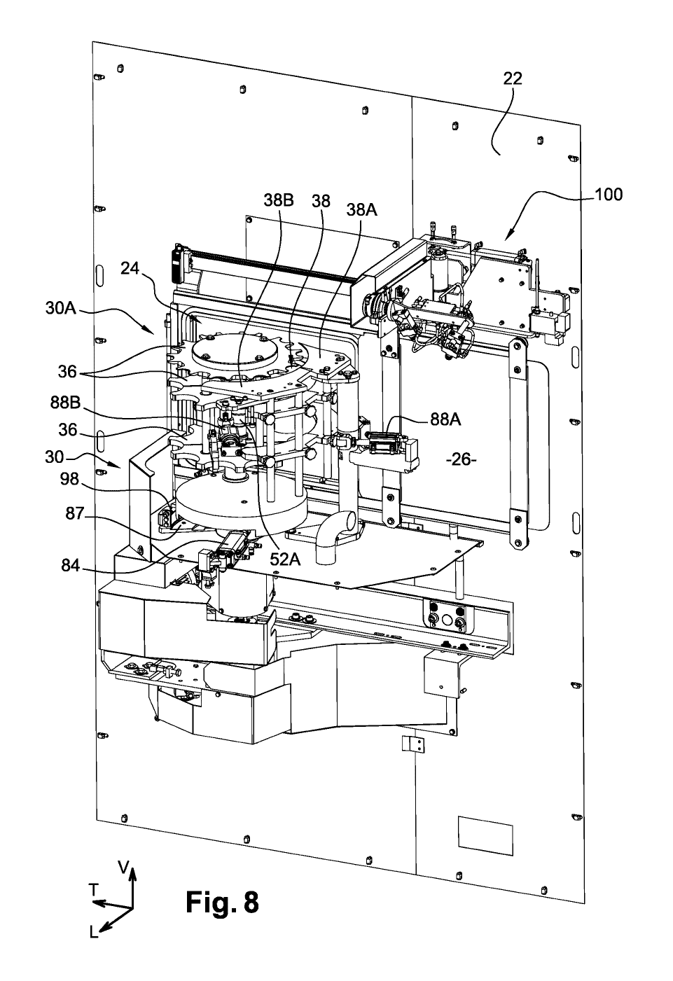

Preferably and as shown in FIG. 7, stop means 95 are provided for ensuring good positioning of the upper part 30A of the wheel 30 for transfer into the transfer position and/or the retracted position.

Advantageously, the stop means 95 make it possible to ensure good vertical positioning of the locking element 92 in relation to the housings 96 and 98.

No later than after the different automatic retraction maneuvers that were just described have been carried out, the closing of the secondary clutch mechanism 70 that switches from the disengaged state to the engaged state is controlled.

In the engaged state, the secondary clutch mechanism 70 ensures the transmission of the torque of the motorized means 80 received by the secondary shaft 54 toward the upper part 30A of the wheel 30 occupying its retracted position owing to which said upper part 30A and more particularly the holding means 36 are driven in rotation during their disinfection by the disinfecting device 100.

Advantageously, the clutch mechanisms 60 and 70 make it possible to carry out automatic re-initiation in order, once the disinfecting operation is performed, to return to the production mode of the facility 10, without requiring in particular the intervention of an operator.

The secondary clutch mechanism 70 is held in the engaged state, or the state that said mechanism 70 occupies during the disinfecting operation for ensuring the driving in rotation of the holding means 36 while the upper part 30A is in the retracted position.

By contrast, the closing of the primary clutch mechanism 60 that switches from the disengaged state to the engaged state, engaged state in which said primary clutch mechanism 60 is still not, however, coupled, is controlled.

As indicated above, the primary clutch mechanism 60 advantageously comprises a torque limiter.

In this stage, the driving of the secondary shaft 54 by the motorized means 80 brings about, with the secondary clutch mechanism 70 still being in the engaged state, the transmission of the torque of the secondary shaft 54 toward the lower part 52B of the primary shaft 52 and this is done by means of the second engaging means 58.

The primary clutch mechanism 60 is then driven, and the torque is detected by detection means, such as a sensor, associated with the torque limiter; a signal is then emitted by the detecting means and used to stop the motorized means 80 and to open the secondary clutch mechanism 70.

FIGS. 8 and 9 show the transfer wheel 30 according to the second embodiment that was just described in detail with reference to FIGS. 5 to 7, with said transfer wheel 30 being illustrated respectively in the transfer position and in the retracted position.

As explained with reference to FIGS. 1 and 2 in particular and illustrated here in FIG. 8, the upper part 30A for transfer of the containers 12 from the wheel 30 extends, in the transfer position, through the opening 24 made in the common part 22 of the first and second enclosures 14 and 18.

As can be seen in FIG. 8, the means 26 for sealing the opening 24 are actually in the open position.

By comparison with FIG. 8 that illustrates the facility 10 in the transfer mode, FIG. 9 illustrates the facility 10 in the decontamination mode.

As illustrated in FIG. 9, the upper part 30A of the transfer wheel 30 was retracted automatically by the actuating means 84; the holding means 36 then no longer extend through the opening 24.

The guide means 38A and 38B are advantageously also retracted automatically by the associated actuators 88A and 88B.

The opening 24 is then free to be closed in an airtight manner by the sealing means 26 that are moved toward their closed position.

The facility 10 is then ready for the implementation of the decontamination operation of the filling unit 20 and also for the disinfecting operation by means of the disinfecting device 100 shown in FIGS. 8 and 9 and more particularly visible in details in FIG. 10.

Below, the disinfecting device 100 will be described according to the second embodiment.

The disinfecting device 100 consists of at least one automatic disinfecting module 110 that, controlled from outside the facility, comprises means 112 for spraying at least one disinfecting agent.

Preferably, said at least one disinfecting agent is formed completely or in part by a compound of the alcohol family, such as ethanol diluted to 70%, which is sprayed in the liquid state by the spraying means 112 of the module 110 for disinfecting at least said upper part 30A for transfer of the containers 12 from the wheel 30.

Preferably, said at least one disinfecting agent is then eliminated naturally by evaporation.

Advantageously, the automatic disinfecting module 110 is able to disinfect at least said upper part 30A for transfer of the containers 12 from the transfer wheel 30.

Preferably, the automatic disinfecting module 110 is designed to disinfect at least the holding means 36, most particularly the upper plate in contact with the neck of the container, and the guide means 38.

Advantageously, the automatic disinfecting module 110 is mounted to move between at least a disinfection position and a rest position.

According to its design and its arrangement in the facility 10, the automatic disinfecting module 110 is mounted to move in translation and/or in rotation to be moved between said disinfecting and rest positions.

The automatic disinfecting module 110 occupies, in the decontamination mode, the disinfection position that corresponds to a position in which said spraying means 112 are able to disinfect at least the upper part 30A for transfer of the containers 12 from the wheel 30.

The automatic disinfecting module 110 occupies, in the production mode, the rest position that corresponds to a position in which the disinfecting device 100 is retracted.

Advantageously, the retraction of the automatic disinfecting device 110 makes it possible in particular to avoid disturbing the flow of air during production when the facility 10 comprises a system for blowing in filtered air to establish overpressure inside the facilities 14 and 18 so as to limit the risks of contamination.

In the retracted position, the automatic disinfecting device 110 is maintained outside of the process followed by the containers 12 in the production mode of the facility 10, which makes it possible to prevent certain risks relative to the containers 12, for example in the case of a leak.

Preferably, and as illustrated in FIGS. 8 and 9, the automatic disinfecting module 110 is connected to said common part 22 of said first and second enclosures 14 and 18.

Advantageously, the automatic disinfecting module 110 is arranged vertically in height in relation to the transfer wheel 30, i.e., vertically above without thereby necessarily being perpendicular to the wheel 30.

Thanks to such an arrangement of the disinfecting module 110 in relation to the transfer wheel 30, said at least one disinfecting agent is in a disinfection position sprayed by said spraying means 112 in the direction of at least the upper part 30A for transfer of the containers 12 from the wheel 30 that is located vertically below.

The automatic disinfecting module 110 comprises at least one arm 114 of said means 112 for spraying said at least one disinfecting agent.

The arm 114 is mounted to move in relation to a frame 116 of the module to be deployed toward the disinfection position or retracted toward the rest position; the stationary frame 116 is connected to the common part 22.

The arm 114 is able to be moved by at least one actuator 118 that is controlled selectively based on the operating, production or decontamination mode of the facility 10.

Preferably, the arm 114 is mounted to move in rotation around a pivot 120 that, determining an axis of rotation A, is integral with the stationary frame 116 of the module 110.

The arm 114 pivots around said pivot 120 between at least one first position corresponding to the disinfection position of the module 110, and a second position corresponds to the rest position of the module 110.

The arm 114 pivots here at 90° in relation to the frame 116 between said first and second positions shown respectively in FIGS. 8 and 9.

Preferably, stop means 122 associated with the actuator 118 determine said first and second positions of the arm 114.

The spraying means 112 are fixed on a support 124 that is advantageously mounted to move in relation to the arm 114 that is moved by the actuator 118 between the disinfection and rest positions.

Preferably, the support 124 extends orthogonally, at 90°, to the free end of the arm 114.

The support 124 is mounted to move in rotation in relation to the arm 114 around an axis B of rotation that is orthogonal to the arm 114 that is itself mounted to move in relation to the frame 116 in rotation around the axis A.

The support 124 that comprises the spraying means 112 is able to be driven in rotation around said axis B by drive means 126, such as a motor.

Advantageously, said means 112 for spraying the disinfecting agent are moved by the support 124 around the axis B, respectively between at least a first angular position P1 and a second angular position P2.

Preferably, the first angular position P1 corresponds to, for example, an angle of 20° in relation to the reference plane at 0° corresponding to a vertical plane of transverse orientation, and the second angular position P2 corresponds to an angle of 10° in relation to said reference plane, said positions P1 and P2 being reached by rotations in the opposite direction in relation to said reference plane.

Advantageously, the rotation of the support 124 makes it possible to carry out, in said disinfection position, a sweeping action during the disinfecting operation of at least said upper part 30A for transfer of the containers 12 from the wheel 30.

Preferably, the stop means 128 are associated with the motor 126 and determine said first angular position P1 and a second angular position P2.

The spraying means 112 of the module 110 forming the disinfecting device 100 comprise at least one nozzle that can spray said at least one disinfecting agent.

As shown in FIGS. 10 to 12, the automatic disinfecting module 110 comprises at least three nozzles respectively referenced 112A, 112B, and 112C.

The position of each nozzle can also be adjusted in at least one direction of the space defined by the trihedron (L, V, T) and in particular adjusted in relation to the support 124.

The nozzles 112A, 112B, and 112C are positioned based on the surfaces to be disinfected, at least one of the nozzles forming, for example, first spraying means for disinfecting said holding means 36 while at least one other nozzle forms second spraying means for disinfecting said guide means 38A and 38B.

FIGS. 11 and 12 more particularly illustrate the jets that are sprayed by said nozzles 112A, 112B, and 112CD that form the spraying means 112 of the module 110.

FIGS. 11 and 12 show the automatic disinfecting module 110 in the disinfection position, the deployed arm 114 is aligned transversely with the frame 116, and the support 124 of the spraying means 112 then extends orthogonally in the longitudinal direction.

In FIG. 11, the support 124 is located in the first angular position P1, i.e., inclined toward the front by an angle of 10° in relation to said vertical reference plane.

In FIG. 12, the support 124 is located in the second angular position P2, i.e., inclined toward the rear by an angle of 20° C. in relation to said vertical reference plane.

In the first angular position P1, the spraying means 112 primarily disinfect the means 36 for holding the transfer wheel 30 while in the second position P2, the spraying means 112 primarily disinfect the guide means 38 formed here from the first guide means 38A and the second guide means 38B.

The disinfecting agent jets sprayed by the nozzles 112A, 112B, and 112C are preferably overall flat jets as FIGS. 11 and 12 illustrate.

During the disinfecting operation carried out automatically with the disinfecting module 110, the upper part 30A of the wheel and therefore the holding means 36 formed by the three superposed plates move relatively with respect to the disinfecting device 100.

As described above, the holding means 36 are advantageously driven in rotation by the motorized means 80 by means of the secondary shaft 54, of the secondary clutch mechanism 70 in the engaged state, and engaging means 56 that act between the upper part 52A of the primary shaft 52 and the secondary shaft 54.

Owing to the driving in rotation of the upper part 30A of the wheel 30, the holding means 36 move relative to the disinfecting device 100, making it possible to obtain a perfect application of said at least one disinfecting agent over the entire holding means 36.

As a variant, not shown, the automatic disinfecting module 110 is mounted to move in translation in relation to the transfer wheel 30, in particular between the rest and disinfection positions.

The module 110 comprises, for example, a carriage that is moved by drive means to slide, parallel to said common part 22 of said first and second enclosures 14 and 18, so as to be advanced or pulled back in relation to the transfer wheel 30.

Advantageously, the spraying means 112 can also be mounted to move, independently of the movement of the disinfecting module 110.

According to the variant, the drive means of the carriage of the automatic disinfecting module 110 are able to drive in movement the means 26 for sealing the opening 24 or conversely in such a way that the movement of said disinfecting module 110 is advantageously carried out simultaneously with the opening and the closing of the opening 24 by the sealing means 26.

1. Facility (10) for producing containers (12), in particular bottles, comprising at least:
a first protective enclosure (14) delimiting a first zone (Z1) inside of which is arranged at least one unit (16) for blow molding containers,
a second confinement enclosure (18) that, at least in the part attached to the first adjacent enclosure (14) by a common part (22), delimits a second sterile zone (Z2) inside of which is arranged at least one unit (20) for filling manufactured containers (12),
at least one opening (24) that, made in said common part (22) of said first and second enclosures (14, 18), is designed to make possible the transfer of the containers (12) from the blow-molding unit (16) to the filling unit (20),
means (26) for sealing said opening (24) that are able to be moved selectively between an open position in which the sealing means (26) allow said transfer of the containers (12) through the opening (24) and a closed position in which the sealing means (26) prevent any transfer by insulating the second enclosure (18) to carry out a decontamination of the filling unit (20),
a transfer device (28) that comprises at least one transfer wheel (30) that is adjacent to said opening (24) for transferring the containers (12) between the blow-molding unit (16) and the filling unit (20), with said transfer wheel (30) comprising at least one upper part (30A) for transfer of the containers (12) that is mounted to move between at least one first transfer position that is occupied when the facility (10) is in a production mode and a second retracted position that is occupied when the facility (10) is in a decontamination mode, with the movement of said upper part (30A) of the wheel (30) mounted to move between said transfer and retracted positions being controlled from outside the facility (10), and
a drive system (50) of the transfer wheel (30) for driving in rotation at least the upper part (30A) for transfer of the containers (12) from the wheel (30) when, in the production mode, said upper part (30A) occupies said transfer position,
wherein the facility (10) comprises a device (100) for disinfecting the transfer wheel (30) that is controlled from outside the facility (10) for disinfecting, by spraying at least one disinfecting agent, at least the upper part (30A) for transfer of the containers (12) from the wheel (30) when, in the decontamination mode, said upper part (30A) for transfer of the containers (12) from the wheel (30) occupies the retracted position.

2. Facility according to claim 1, wherein the disinfecting device (100) consists of at least one gun (105) arranged inside the facility (10) and able to be operated by an operator from outside the facility, by means of remote handling means (55), for spraying said at least one disinfecting agent on at least said upper part (30A) for transfer of the containers (12) from the wheel (30).

3. Facility according to claim 1, wherein the disinfecting device (100) consists of at least one automatic disinfecting module (110) that, controlled from outside the facility (10), comprises means (112) for spraying said at least one disinfecting agent to disinfect at least said upper part (30A) for transfer of the containers (12) from the wheel (30).

4. Facility according to claim 3, wherein said automatic disinfecting module (110) is mounted to move between at least:
a disinfection position, occupied in the decontamination mode, in which said spraying means (112) are able to disinfect at least the upper part (30A) for transfer of the containers (12) from the wheel (30); and
a rest position, occupied in the production mode, in which said disinfecting module (110) is retracted.

5. Facility according to claim 4, wherein the automatic disinfecting module (110) comprises at least one arm (114) of said means (112) for spraying the disinfecting agent and wherein said arm (114) is moved selectively by at least one actuator (118) to be deployed toward the disinfection position or retracted toward the rest position.

6. Facility according to claim 4, wherein said means (112) for spraying the disinfecting agent are mounted to move in rotation around an axis (B), respectively between at least a first angular position and a second angular position, for carrying out in said disinfection position a sweeping action of at least said upper part (30A) for transfer of the containers (12) from the wheel (30).

7. Facility according to claim 4, wherein said upper part (30A) for transfer of the containers (12) from the wheel (30) comprises at least the holding means (36) with which are associated the guide means (38) that, in the production mode, participate respectively in the transfer of the containers (12), and wherein the automatic disinfecting module (110) comprises at least the first spraying means for disinfecting said holding means (36) and the second spraying means for disinfecting said guide means (38).

8. Facility according to claim 1, wherein said at least one disinfecting agent is formed completely or in part by a compound of the alcohol family that is sprayed in the liquid state by the disinfecting device (100) for disinfecting at least said upper part (30A) for transfer of the containers (12) from the wheel (30).

9. Facility according to claim 1, wherein at least one part of the disinfecting device (100) or the upper part (30A) for transfer of the containers (12) from the wheel (30) occupying said retracted position is able to carry out one relative movement in relation to the next to perfect the application of said at least one disinfecting agent.

10. Facility according to claim 1, wherein said drive system (50) of the transfer wheel (30) is able to drive in rotation at least said upper part (30A) for transfer of the containers (12) from the wheel (30) when, in the decontamination mode, said upper part (30A) for transfer of the containers (12) occupies said retracted position.

11. Facility according to claim 10, wherein said drive system (50) of the transfer wheel (30) of the containers (12) comprises at least one primary shaft (52) made in at least two parts, respectively a first upper drive part (52A) to which is connected at least said upper part (30A) for transfer of the containers (12) and a second lower drive part (52B), said first and second parts (52A, 52B) being linked in rotation to a secondary shaft (54).

12. Facility according to claim 11, wherein the first and second parts (52A, 52B) of the primary shaft (52) are respectively linked in rotation to the secondary shaft (54) by mating shapes; preferably, said connection by rotation is made by engagement.

13. Facility according to claim 11, wherein a secondary clutch mechanism (70) is associated with the secondary shaft (54) to control its driving selectively by motorized means (80).

14. Facility according to claim 13, wherein said secondary clutch mechanism (70) occupies at least:
an engaged state when said at least one upper part (30A) for transfer of the containers (12) from the wheel (30) is in the retracted position for coupling in rotation, by means of the secondary shaft (54), said upper part (52A) of said primary shaft (52) to said motorized means (80) so as to drive in rotation said upper part (30A) for transfer of the containers (12), and
a disengaged state when said at least one upper part (30A) for transfer of the containers (12) from the wheel (30) is in the transfer position to disconnect said secondary shaft (54) from said motorized means (80).

15. Facility according to claim 5, wherein said means (112) for spraying the disinfecting agent are mounted to move in rotation around an axis (B), respectively between at least a first angular position and a second angular position, for carrying out in said disinfection position a sweeping action of at least said upper part (30A) for transfer of the containers (12) from the wheel (30).

16. Facility according to claim 5, wherein said upper part (30A) for transfer of the containers (12) from the wheel (30) comprises at least the holding means (36) with which are associated the guide means (38) that, in the production mode, participate respectively in the transfer of the containers (12), and wherein the automatic disinfecting module (110) comprises at least the first spraying means for disinfecting said holding means (36) and the second spraying means for disinfecting said guide means (38).

17. Facility according to claim 6, wherein said upper part (30A) for transfer of the containers (12) from the wheel (30) comprises at least the holding means (36) with which are associated the guide means (38) that, in the production mode, participate respectively in the transfer of the containers (12), and wherein the automatic disinfecting module (110) comprises at least the first spraying means for disinfecting said holding means (36) and the second spraying means for disinfecting said guide means (38).

18. Facility according to claim 12, wherein a secondary clutch mechanism (70) is associated with the secondary shaft (54) to control its driving selectively by motorized means (80).
